(19) United States
(12) Patent Application Publication
(10) Pub. No.: US 2003/0215426 A1
(43) Pub. Date: Nov. 20, 2003

(54) Redifferentiated cells for repairing cartilage defects

(75) Inventors: Margaret M. French, Houston, TX (US); Kyriacos A. Athanasiou, Houston, TX (US)

Correspondence Address: CONLEY ROSE, P.C., P. O. BOX 3267, HOUSTON, TX, 77253-3267, US

(73) Assignee: William Marsh Rice University, Houston, TX

(21) Appl. No.: 10/405,287

(22) Filed: Apr. 2, 2003

Related U.S. Application Data
(60) Provisional application No. 60/369,421, filed on Apr. 2, 2002.

(51) Int. Cl. A61K 45/00; C12N 5/08

(52) U.S. Cl. 424/93.7; 435/366

(57) ABSTRACT

A redifferentiated dermal fibroblast cell that exhibits at least one characteristic of a chondrocyte. A proteoglycan is used to induce re-differentiation of the cell. In some embodiments, the cell expresses of at least one cartilage proteoglycan marker. The proteoglycan may comprise aggrecan and the cell may differentiate from the fibroblast along the chondrogenic lineage.

A method of inducing chondrogenesis in a fibroblast cell comprises culturing the fibroblast cell on a surface containing at least one cartilage-derived proteoglycan other than perlecan. A three-dimensional scaffold may be coated with the proteoglycan and seeded with fibroblast cells. The fibroblast cells may be contacted with at least one chondrogenic growth factor or cytokine prior to said culturing.

### CROSS-REFERENCE TO RELATED APPLICATIONS

The present invention claims priority to U.S. Provisional Application Serial No. 60/369,421, filed Apr. 2, 2002, and entitled “Redifferentiation of Differentiated Cells to Chondrocytes,” which is incorporated herein in its entirety.

### STATEMENT REGARDING FEDERALLY SPONSORED RESEARCH OR DEVELOPMENT

Not Applicable.

### BACKGROUND OF THE INVENTION

1. Field of the Invention

The present invention generally relates to the repair of cartilage damage or defects, and more particularly to compositions and methods for the production of implantable cartilage-like products. Still more particularly, the present invention relates to the generation of such products using in vitro redifferentiation of fibroblasts on a cartilage matrix proteoglycan.

2. Description of Related Art

Articular cartilage is a low friction, durable material that is present in animal joints. Cartilage distributes mechanical forces within the joint and protects the underlying bone. Despite this important function, cartilage is avascular and therefore virtually incapable of healing or repairing itself adequately in response to trauma or pathology. Injuries to articular cartilage, and to knee joints in particular, are common. Because the cartilage does not heal, injuries tend to remain, or even progress over time. Hence there is an ongoing need for a technique or composition that can be used to repair torn or damaged articular cartilage.

Articular cartilage consists of chondrocytes dispersed in an extracellular matrix. Chondrocytes are specialized cells that produce and maintain the matrix. The chondrocytes are thinly distributed in the matrix, however, and are not present in sufficient quantities to rebuild the matrix or repair injuries to it. Likewise, cellular migration from the vascular system does not occur with pure chondral injury and extrinsic repair is clinically insignificant.

The physical properties of articular cartilage are largely the result of the molecular structures of type II collagen and aggrecan, which are components of the extracellular matrix, along with hyaluronan and type IX collagen. Type II collagen forms a 3-dimensional network or mesh that provides the tissue with high tensile and shear strength. Aggrecan is a large, hydrophilic molecule, which is able to aggregate into complexes comprising thousands of units. Aggrecan molecules contain glycosaminoglycan chains comprising large numbers of sulfate and carboxylate groups. In cartilage, aggrecan complexes are entrapped within the collagen network.

Because the naturally occurring repair mechanisms are insufficient, researchers have proposed various in vitro approaches to the production of cartilage tissue. These typically involve seeding cultured cells (either chondrocytes or pluripotential stem cells) into a biological or synthetic scaffold. A primary difficulty with these approaches is the shortage of suitable cells for culturing or seeding.

Mesenchymal stem cells, for example, are pluripotential and have shown promise in the field of tissue engineering and regeneration. Isolated from a variety of adult tissues such as bone marrow, processed liposuction waste, and patellar fat pad, these cells can give rise to a variety of new cell types. These differentiation pathways are becoming clearer with time, and the variety of cell types that can be obtained is ever expanding. Although the bone marrow stem cells may be the best characterized of the stem cells, they are not easily obtained and comprise only a small percentage of the population of marrow isolate. While few people may object to having fat removed for use in repair of other parts of the body, the procedure is nonetheless invasive. The collection of patellar fat pad cells can be performed using arthroscopy, but it too is an invasive procedure that yields only small numbers of cells. Thus, the versatility of stem cells is outweighed by the current difficulty of obtaining them. Major drawbacks of previous approaches are: (1) limited availability of either chondrocytes or pluripotential stem cells; and (2) difficulty in attaining or maintaining the chondrocyte phenotype.

It has been demonstrated by French et al. (J. Cell Biol. (1999) 145: 1103-1115) that multipotential 10T1/2 murine embryonic fibroblast cells grown on the proteoglycan perlecan, but not on a variety of other matrices, stimulated extensive formation of dense nodules resembling embryonic cartilaginous condensations. In those studies, perlecan was found to be not only a marker of chondrogenesis, but also a strong potentiator of chondrogenic differentiation in vitro. Other extracellular matrix molecules and glycosaminoglycans failed to induce differentiation. It was also observed that human fibroblasts did not attach to or differentiate on perlecan-coated surfaces. Human chondrocytes, however, maintained their differentiated form in vitro when cultured on a perlecan-coated surface.

If one were to characterize the ideal cell to use for tissue regeneration, it would be a cell that proliferates rapidly, is easy to obtain from the patient and can be maintained in a differentiated state such that a mimic of the desired tissue can be generated. Because none of the techniques proposed to date for producing cartilage-like tissue is entirely satisfactory, a need remains for a technique that can be used to generate sufficient numbers of chondrocytes using cells having these desired characteristics. An implantable tissue engineered structure that is capable of functioning in the body as articular cartilage is especially needed by the medical community.

### SUMMARY OF THE INVENTION

The present invention avoids the obstacles associated with the use of stem cells for tissue engineering. At the same time it takes advantage of abundant but fully differentiated fibroblasts as the source of cells for cartilage repair. The methods described herein allow cells that have already differentiated to be redifferentiated into chondrocytes on demand. More specifically, the present invention provides a technique whereby fibroblasts, or other differentiated cells, are cultured in the presence of a cartilage matrix protein and thereby caused to redifferentiate into chondrocytes. The newly-formed chondrocytes are fully functional, producing aggrecan and other biochemicals that are characteristic of chondrocytes. Hence, the present invention provides a technique for generating large numbers of chondrocytes having sufficient activity to allow effective repair and replacement of injured or damaged cartilage.

A particular embodiment entails the use of aggrecan as a bioactive agent to assist differentiated fibroblasts exhibit cartilage-like behavior and thus produce extracellular matrix components specific of articular cartilage.

In certain embodiments, a redifferentiated fibroblast cell exhibits at least one characteristic of a chondrocyte, when a proteoglycan other than perlecan is used to induce redifferentiation of that cell. The differentiated cell that is redifferentiated into a chondroctye may be a dermal fibroblast cell. The chondrocytic characteristic expressed by the redifferentiated cell may be expression of collagen type II and/or mRNA encoding of said collagen type II. The chondrocytic characteristic may comprise expression of at least one cartilage proteoglycan marker and may in particular comprise a glycosaminoglycan. The proteoglycan preferably comprises aggrecan. The present cells are believed to differentiate along the chondrogenic lineage. Cells redifferentiated according to the present invention can be used to create a composition for treatment of a cartilage defect or disorder, which may in some instances comprise a three-dimensional structure.. Alternatively, cells redifferentiated according to the present invention can form part of a kit for treatment of a cartilage defect. The cells can be allogenic or autologous to an individual in need of such treatment.

A preferred method for making redifferentiated cells according to the present invention comprises culturing cells on a surface coated with a proteoglycan other than perlecan. The cells may be dermal fibroblast cells, or other differentiated cells, including chondrocytes. If desired, a three-dimensional scaffold can be coated with the proteoglycan and seeded with fibroblast cells. The proteoglycan is preferably aggrecan. The method can include treating the fibroblast cells with at least one growth factor or cytokine, such as IGF-1.

Differentiated cells that are suitable for use in the present invention include fibroblasts, muscle cells, fat cells, tendon cells, ligament cells and chondrocytes.

The present invention comprises a combination of features and advantages that enable it to overcome various problems of prior devices. The various characteristics described above, as well as other features, will be readily apparent to those skilled in the art upon reading the following detailed description of the preferred embodiments of the invention, and by referring to the accompanying drawings.

### BRIEF DESCRIPTION OF THE DRAWINGS

For a more detailed description of the preferred embodiment of the present invention, reference will now be made to the accompanying drawings, wherein:

FIG. 1 is a flow diagram illustrating one preferred embodiment of the present invention.

FIGS. 2A and 2B are photomicrographs of a fibroblast seeded scaffold cultured in a bioreactor. FIG. 2A was taken at the start of culturing. FIG. 2B was taken after culturing for six weeks.

FIGS. 3A and 3B are photomicrographs of rabbit skin cells cultured in accordance with an embodiment of the present invention and stained to reveal proteoglycans. A. Cells grown on aggrecan for 24 hours; B. Control cells; C. Cells grown on aggrecan for one week; D. Control cells after one week; E. Aggrecan grown cells after four weeks; F. Control cells after four weeks. (A, B—100×; C-F—200× magnification)

FIGS. 4A-D are photomicrographs of adult rabbit skin cells cultured in accordance with an embodiment of the present invention and stained to reveal proteoglycans (100× magnification)

FIGS. 5A-D are photomicrographs showing the morphology of human foreskin cells cultured for 24 hours in accordance with one embodiment of the present invention. A. Control cells (40× magnification); B. Aggrecan treated cells (40×); C. Aggrecan treated cells (200×); D. Aggrecan treated cells (200×).

FIGS. 6A-C are photomicrographs showing proteoglycan staining of human foreskin cells cultured for one week, in accordance with one embodiment of the invention. A. Control cells (100× magnification); B. Cells grown on aggrecan (100×); C. Cells grown on aggrecan (200×).

FIGS. 7A-D are photomicrographs showing proteoglycan staining of human foreskin cells cultured for one week, in accordance with one embodiment of the invention. A. Control cells (100× magnification); B. Cells grown on aggrecan (100×); C. Cells grown on aggrecan (100×); D. Cells grown on aggrecan (200×).

FIGS. 8A-D are photomicrographs showing collagen type II staining of human foreskin cells cultured for one week, in accordance with one embodiment of the invention. A. Control cells; B. Cells grown on aggrecan; C. Cells grown on aggrecan; D. Cells grown on aggrecan. (All 100× magnification).

### DETAILED DESCRIPTION OF THE PREFERRED EMBODIMENTS

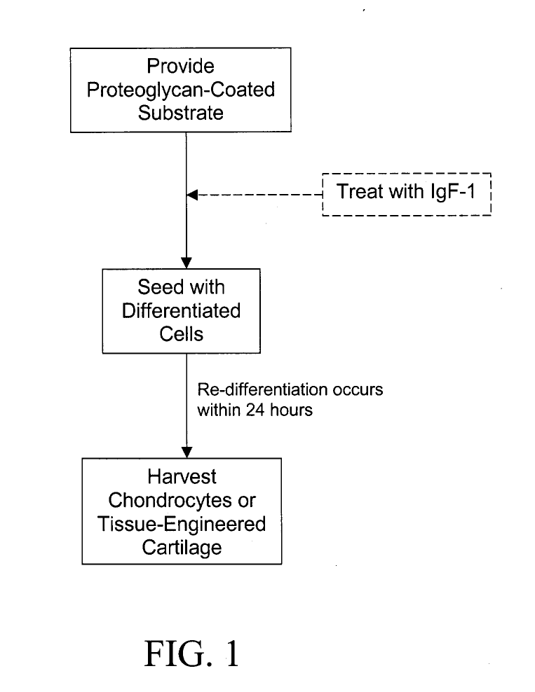

The present invention provides a cartilage regeneration and repair technique that induces differentiated cells to differentiate into cartilage tissue, a product comprising such differentiated cells, and methods of using such a product to repair cartilage lesions or defects. Referring initially to FIG. 1, a flow diagram of a process for converting fibroblasts into cartilage-like cells and for forming an implantable tissue engineered construct for use in repairing an articular cartilage defect is shown. The basic method includes the steps of (a) providing a substrate for the cells to attach to and grow on, (b) coating the substrate with one or more proteoglycans, (c) seeding the coated substrate with precursor or progenitor cells, preferably dermal fibroblasts such as human foreskin, and (d) allowing the precursor cells to differentiate into chondrocytes. The steps may be carried out on a scaffold if desired, and the proteoglycan(s) may be incorporated into the scaffold or substrate, instead of or in addition to being present as a surface coating. The chondrocytes formed in this manner and the associated extracellular matrix secreted by those cells are harvested and can be implanted as tissue-engineered cartilage (e.g., resurfacing an articular cartilage defect).

This process can be carried out either on a three-dimensional structure, such as a scaffold, or in a two-dimensional environment, such as a culture dish. Both embodiments are discussed in detail below. One preferred process for constructing a three-dimensional tissue-engineered construct preferably includes:

(a) obtaining a suitable scaffold material (having sufficient mechanical integrity and including appropriate bioactive agents or growth factors, preferably at least one proteoglycan);

(b) seeding the scaffold with chondrogenic cells, preferably dermal fibroblasts;

(c) culturing the seeded scaffold in a bioreactor while subjecting the structure to conditions which promote formation of the desired tissue engineered construct (e.g., mechanical stimulation including hydrostatic pressure, direct compression or other biomechanical bioreactor effects, and favorable biochemical and nutrient transport conditions); and then

(d) surgically replacing the cartilage defect with the resulting tissue engineered construct. An optional preliminary step comprises determining the characteristics of native articular cartilage (e.g., biomechanical, biochemical and cellular features) so as to enable the provision of a tissue-engineered cartilage that more closely resembles the desired tissue.

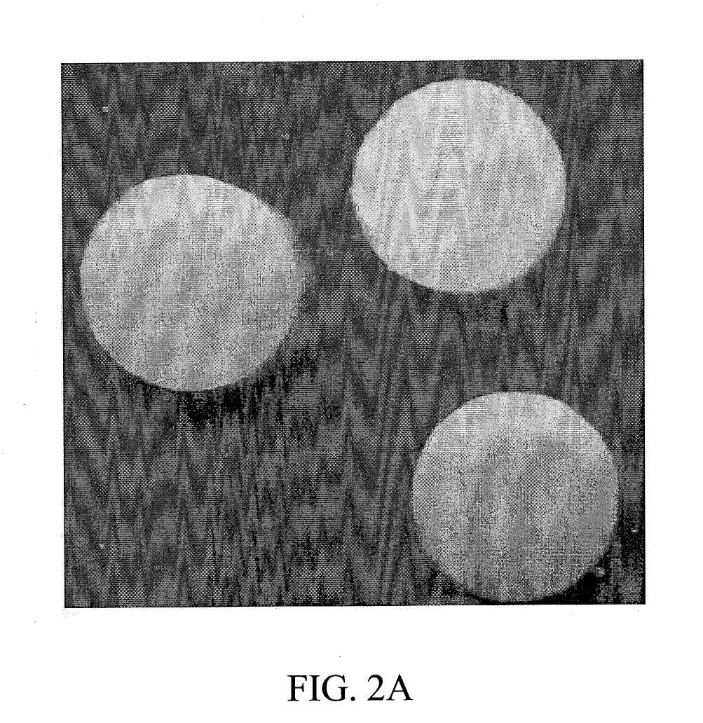

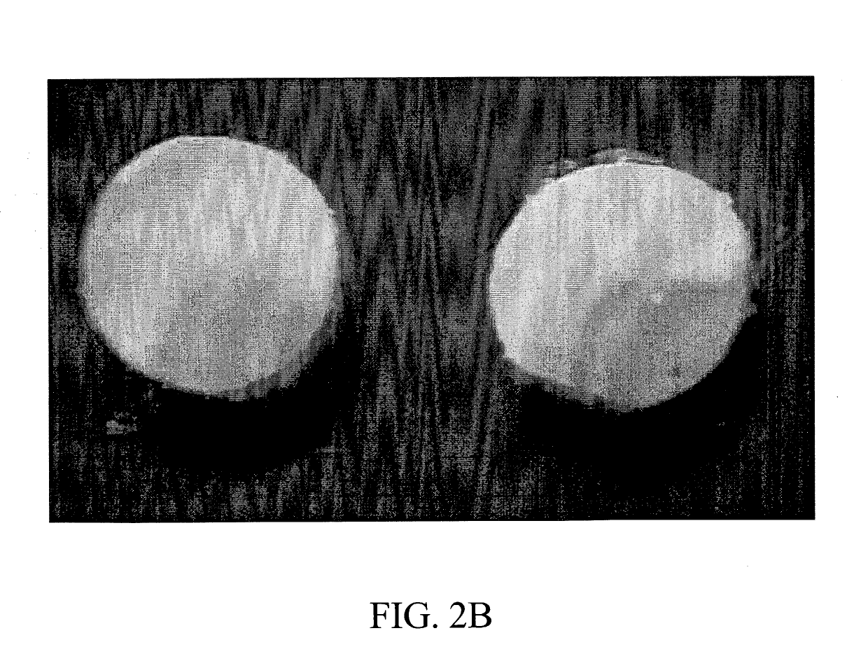

FIG. 2A is a photomicrograph showing a representative cell seeded scaffold upon commencement of culturing. FIG. 2B shows the same structure after six weeks of culture. In FIG. 2A, the scaffolds are clean and well-defined. In the second picture, nodules of cells are visible on the scaffolds as well as a loose structure of cells around the scaffold. After six weeks, the cells have proliferated and formed a pre-cartilaginous structure, i.e. a nodule (or aggregate). In addition, testing confirms that after six weeks the cells are producing ECM. The materials and methods employed to make this construct are described in more detail below.

In addition to the above-described three-dimensional construct, other highly desirable products for cartilage repair are prepared under two-dimensional culture conditions as exemplified below.

Substrates

Materials that are suitable for use as substrates for the tissue culture cells include, but are not limited to, polymers; biodegradable polymers; hydrogels, ceramics, composites, and natural materials such as collagen. For in vitro redifferentiation, the substrate can be as simple as a standard tissue culture dish. In other embodiments, such as for example when it is desired to provide an implantable tissue-engineered device, the substrate may be preformed as a three-dimensional scaffold having the desired ultimate shape, or may be shaped while the cells proliferate in a bioreactor, as illustrated in FIG. 1. The scaffold can comprise a porous or non-porous structure, and is preferably a biodegradable polymer. It is desirable to select a scaffold material that has sufficient mechanical integrity to withstand the loading and stresses that will be imposed on it and is capable of completely dissolving or degrading as the proliferating cells fill and assume the shape of the scaffold. Such scaffold materials are well known in the art and have been described in the literature.

Proteoglycans

Once the desired substrate is selected, it is coated with a solution of one or more proteoglycans and allowed to dry. Proteoglycans that are suitable for use in the present invention include particularly cartilage-derived proteoglycans selected from the group consisting of aggrecan, perlecan, decorin biglycan, proteoglycan aggregates, proteoglycan monomers, link proteins, aggrecan aggregates, aggrecan monomers, hyaluronic acid, and mixtures thereof. Preferably the proteoglycan is aggrecan. The proteoglycan is suspended in phosphate buffered saline (PBS) or any other suitable carrier. An effective amount of the aggrecan solution is added to the tissue culture vessel and allowed to dry such that the cell contacting surface(s) are coated with aggrecan.

Precursor Cells

Differentiated cells that can be redifferentiated using the present technique include but are not limited to fibroblasts, muscle cells, fat cells, tendon cells, ligament cells and cells from other types of cartilage. Preferably dermal fibroblasts are used because they proliferate rapidly, are easy to obtain from the patient or from an allogenic donor, and can be maintained in a differentiated state as desired. Adult or neonatal fibroblasts can be used, although in some instances adult cells may require the presence of a growth factor such as IGF-1, as described below, to bring about redifferentiation. One preferred donor source for allogenic fibroblasts is human foreskin tissue. Although less preferred, stem cells, embryonic fibroblasts, and other multipotential mesenchymal cells will also differentiate under the disclosed culture conditions to yield chondrocytes or chondrycyte-like cells. Other differentiated cell lines that are suitable for use in the present technique include embryonic fibroblasts and the cell lines identified above.

It has also been found that the present techniques are surprisingly effective for culturing chondrocytes. Previously, attempts to culture chondrocytes with traditional cell culture methods resulted in the chondrocytes returning to an undifferentiated cell type. When cultured on aggregan, however, chondrocytes grow and secrete ECM indefinitely.

Treatment with IGF

In some instances, it is desirable to pretreat the cells with a growth factor, and in particular with a chondrogenic growth factor. In these instances, IGF-1 is preferably added to the media every 48 hours. While some newly-harvested cells, including adult dermal fibroblasts, redifferentiate readily to chondrocytes, IGF-1 can cause or enhance redifferentiation in cell types that do not differentiate as readily, adult dermal fibroblasts and cell cultures having higher passage numbers. Alternatively, one or more other suitable growth factor that is capable of assisting in the redifferentiation process could be used instead of, or in addition to, IGF-1.

Redifferentiation

The precursor cells are cultured on the coated substrate for at least four hours, more preferably for at least 12 hours, and still more preferably for at least 24 hours. If it is determined that the chondrogenic process is further promoted by the addition of one or more growth factors, such as IGF-1, to the culture medium, they may be added intermittently throughout the culturing process.

The resulting cells may be examined for expression of chondrocyte markers and for their morphological resemblance to chondrocytes. Whether the precursor cells adequately redifferentiated along a chondrocytic pathway during culture period is preferably determined by examining the cell morphology after 12-24 hours. If tissue culture is continued beyond the initial 24 hour period, maintenance of the chondrocyte phenotype can be monitored by identification of the production of proteoglycans, collagen type II and expression of marker genes.

### EXAMPLES

The following examples are provided to illustrate preferred embodiments of the invention and do not limit the claims in any way.

### Example 1

Redifferentiation of Rabbit Dermal Fibroblasts to Cartilage-Like Cells Cell Differentiation Assay

An adult rabbit dermal fibroblast cell line, Rab9, was obtained from American Type Culture Collection (ATCC, Manassas, Va.). Cells were maintained in DMEM with 10% fetal bovine serum, 1% pen-strep (Gibco/Invitrogen, Carlsbad, Calif.) and 1% Fungizone (Gibco/Invitrogen, Carlsbad, Calif.). IGF-I was obtained from Diagnostic Systems Laboratory (Houston, Tex.). For pretreatment of Rab9 cells, 10 ng IGF-I/ml media was added to the media every 48 hours. The differentiation assays were similar to those described by French et al. (1999), which is hereby incorporated herein by reference. Briefly described, 24 well plates were coated with 5 μg aggrecan (Sigma, St. Louis, Mo.), resuspended in PBS and sterile filtered prior to use. Aggrecan was added to the well and PBS was subsequently added to make a final volume of 300 μl. Plates were allowed to dry overnight at 37° C. On day two, wells were rinsed with PBS prior to plating. 2×10⁵ cells were plated per well in 0.5 ml of media. Media was changed every other day during assays.

Morphologic Examination

Formation of cell aggregates during culture was assessed by visual examination using a microscope. Cells that had drawn together indo dense, multi-layered regions resembling condensed mesenchyme of developing cartilage, leaving areas of the well bare, were scored as aggregate positive. Also, cells in these regions were highly rounded compared with the fibroblastic morphology typical of Rab9 cells.

Safranin O Staining

At each time point, media was carefully removed from the wells and cells were washed with PBS. After a 10 minute fixation in formalin, cells were rinsed with water and stained with Fast Green for 10 minutes. After the water wash, a brief incubation in acetic acid was performed. Immediately following the acid, Safranin O was added to the wells for 2 minutes. After a water rinse, cells were photographed using a Nikon CoolPix™ 990 digital camera mounted on a Nikon Eclipse TS-100 inverted microscope.

Collagen Type II Staining

Wells were rinsed with PBS, blocked with 1:50 dilution of goat serum in PBS for 1 hour at room temperature, incubated with the primary antibody overnight at 4° C., washed with PBS prior to incubation of biotinylated 2° antibody for 40 min at 37° C. Controls were incubated with PBS overnight in place of primary antibody. The color reaction was carried out following the manufacturer's protocol for the VectaStain® reagents (Vector Laboratoriess, Inc., Burlingame, Calif.).

RNA Isolation and Reverse Transcriptase-Polymerase Chain Reaction

RNA was isolated using an Ambion RNAqueous™ kit (Ambion, Inc., Austin, Tex.) following the manufacturer's instructions. Lysis buffer provided in the kit was added to rinsed cells in the wells. The wells were scraped with the pipet tip to ensure complete lysis and cell collection. Samples were processed through the RNA isolation spin columns in accordance with the manufacturer's instructions. Elution was achieved in two steps using 30 μl of elution buffer. RNA was treated with DNase for 15 minutes at 65° C., followed by heating at 95° C. for 10 minutes. RNA was stored at −80° C. prior to use for RT reactions. The RT reaction contained SuperScript RT™ (Strategene, Hercules, Calif.), the provided buffer, 10 U RNase Inhibitor (Promega, Madison, Wis.), 2.5 μmol random hexamer primers, 10 μM dNTPs, RNA and DEPC water to a final volume of 20 μl. The reaction proceeded at 42° C. for 60 minutes. After completion of the RT reaction, samples were either stored at −20° C. or used immediately for PCR. For the PCR reaction, 2.5 U Fisher Taq polymerase was used per reaction with the provided buffer. Primers were used at a concentration of 2 mM unless otherwise specified. Between 2-5 ml of cDNA was added to each reaction and dNTPs were added at a concentration of 1 mM. Thermocycling was carried out on a Perkin Elmer GeneAmp 4600 machine with a cycling protocol of 5 minutes initial denature, 25 cycles of 30 seconds at 95° C., 30 seconds at 55° C., 1 minute at 72° C., followed by a 10 minute extension. Products were identified by agarose gel electrophoresis on a 2% gel. GelExpert™ Imaging software (NucleoTech, San Carlos, Calif.) was used to photograph the gels.

Dimethylmethylene Blue Assay

Samples were digested in the well for 10 hours with 200 μl papain in 0.5M acetic acid per well. After digestion, samples were transferred to 1.5 ml centrifuge tubes. For the dimethylmethylene blue (DMMB) assay, 50 μl was tested from each sample. Glycosaminoglycan (GAG) concentration was determined using a Blyscan™ kit with provided standards. Briefly, the sample is incubated with the DMMB dye. The ensuing precipitate was centrifuged and the supernatant removed. The pellet was resuspended in a dissociation buffer and the OD550 of the solution was obtained. Statistical data was obtained using StatView™ software.

Hydroxyproline Assay

To determine total collagen amounts in each sample, samples were digested in papain, as described above. 100 μl of each sample was added to a total volume of 1 ml 6 M HCl (final concentration) and incubated overnight at 115° C., or until all liquid had evaporated. Samples were resuspended in Chloramine-T reagent (1.14 g Chloramine-T dissolved in 20.7 ml water, mixed with 26 ml isopropanol and 53.3 ml 1× Stock Buffer [10× buffer: 50 g citric acid, 12 ml glacial acetic acid, 120 g sodium acetate and 34 g sodium hydroxide for a total volume of 1 L]), incubated at room temperature for 20 minutes, protected from light. After incubation with Chloramine-T, 1 ml of freshly prepared dimethylaminobenzaldehyde reagent (15 g dimethylaminobenzaldehyde was suspended in isopropanol to form a slurry, 26 ml of 60% perchloric acid was added slowly) was added, vortexed briefly and incubated at 60° C. for 15 minutes. Samples were cooled in water for five minutes prior to reading absorbance at 550 nm.

Results and Discussion

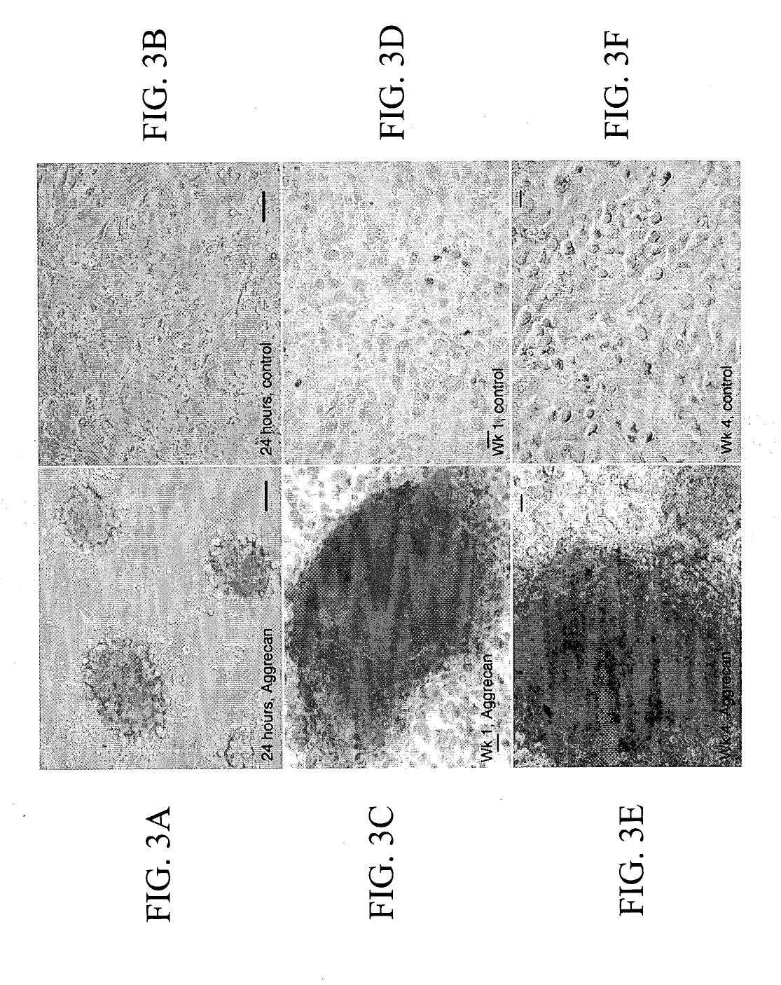

To test the differentiation potential of the adult rabbit dermal fibroblast cell line Rab9, an assay similar to that previously employed with mouse embryonic cell line 3T3/10T1/2 (French (1999)) was attempted. Rab9 cells were plated on either an aggrecan-coated plastic tissue culture surface or directly on the tissue culture plastic. In the initial assay, the Rab9 cells on aggrecan failed to show any signs of differentiation. When the cells were pretreated with IGF-1 as described above, however, the Rab9 cells plated on aggrecan exhibited a much stronger cell growth response. As shown in FIG. 3A, after 24 hours on aggrecan, the IGF-1 treated Rab9 cells are almost exclusively in compact clusters, as compared to the monolayer of cells seen in culture on uncoated tissue culture plastic (FIG. 3B). Thus, while the initial cultures failed to show any morphological change in culture on aggrecan, pre-exposure to IGF-1 results in a dramatic change in cell shape in the aggrecan assay.

To confirm that the differentiation of the Rab9 cells was along the chondrogenic lineage, several assays were performed to examine cartilage marker expression. As chondrocytes synthesize large amounts of proteoglycan, namely aggrecan, staining with the ionic dye, Safranin O, was performed. FIGS. 3C and 3E show the intense staining of the aggregates seen with Safranin O at the 1 and 4 week time points, respectively. By contrast, cultures on plastic were negative (FIGS. 3D and 3F).

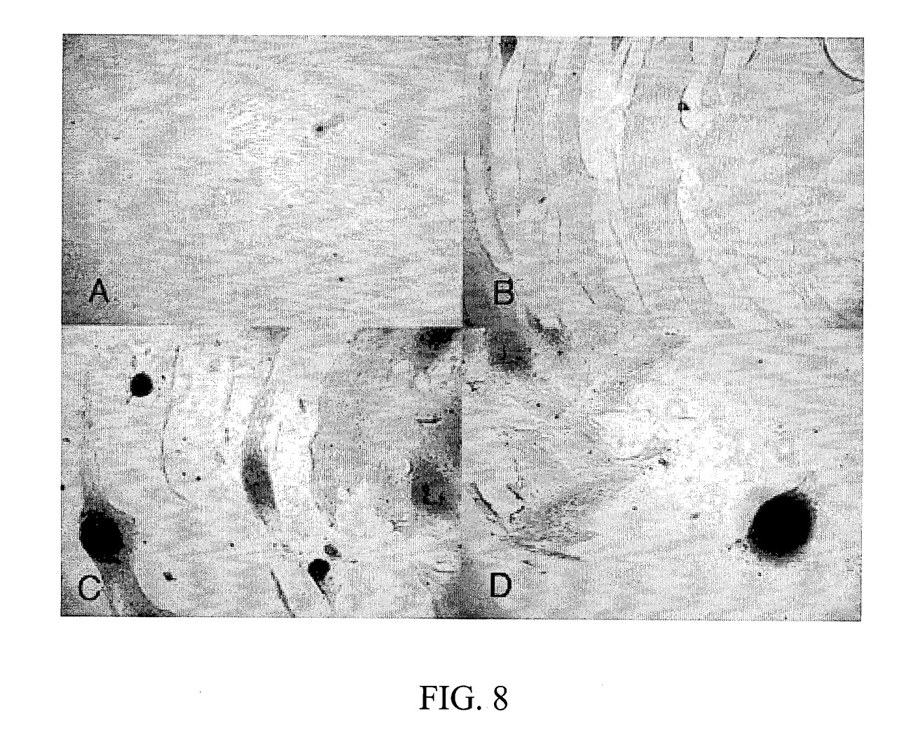

To confirm that these cells were secreting new aggrecan, and not simply recycling that which was coating the culture surface, examination of mRNA expression by RT-PCR was performed and showed that these cells were synthesizing aggrecan. In conjunction with the expression of the matrix proteoglycan, aggrecan, mRNA for collagen type II was also detected. The mRNA expression translates into protein as detected by an antibody against collagen type II. Strong staining of the aggregates was also seen with the antibody to collagen type II (FIG. 8). This signal is specific for the antibody as aggregates, incubated under the same conditions but lacking primary antibody, show little to no staining.

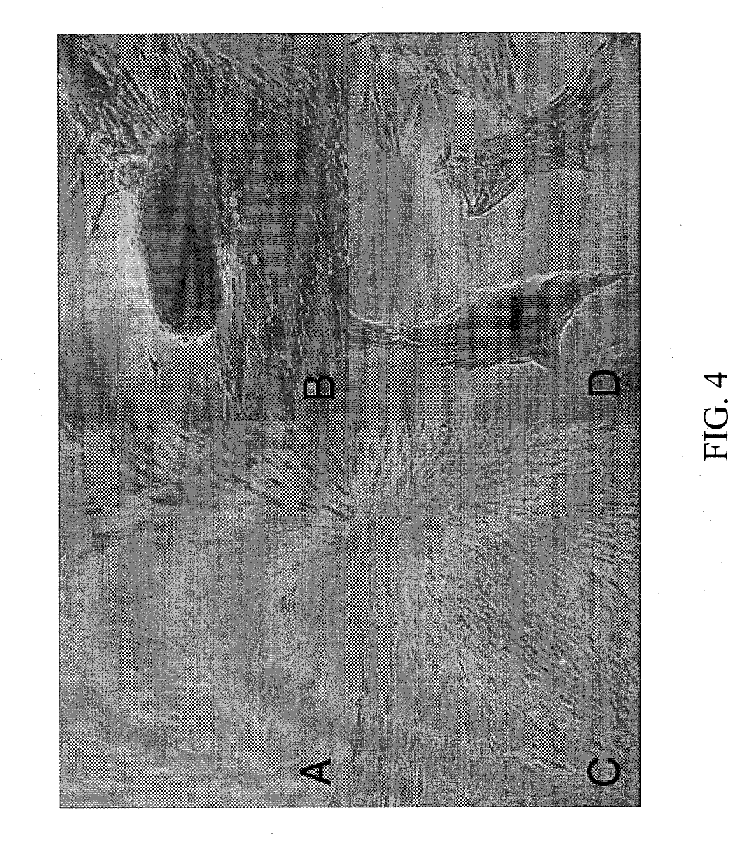

Chondrocytes characteristically express higher amounts of proteoglycans than do fibroblastic cells. Thus, total glycosaminoglycan (GAG) concentration was determined for cells on aggrecan or on plastic. The GAG values were normalized to amounts of DNA as determined by PicoGreen® assay (Molecular Probes, Inc., Eugene, Oreg.). While the normalized data did not show significantly different amounts of proteoglycan in the cultures on aggrecan, this is most likely due to the aggregates being a small contributor to the cell population of the culture as a whole. It can be shown by histochemical staining that there are elevated levels of proteoglycans in the aggregates. This is seen in the assay for proteoglycans using Safranin O as described above. FIGS. 4A and 4C show the control cultures on plastic at week 1 and week 4, respectively, while FIGS. 4B and 4C show the aggregates formed by the same cells in the same time period when cultured on aggregan. The cells on plastic do express proteoglycans, although these are probably basement membrane proteoglycans rather than cartilage matrix proteoglycans.

In addition to examining proteoglycan production, total collagen synthesis was also measured by hydroxyproline assay. As this assay is not specific for the various collagen types, collagen type I can dominate the other types. After normalization to DNA values, the contribution of type I collagen to the combined measure was readily apparent as the monolayer control cultures showed increasing amounts of collagen that were statistically different from the aggrecan cultures. However, this difference clearly shows the distinction between the cultures. While the cells on aggrecan synthesize collagen type II, their numbers are such a small percentage of the control culture that the values are significantly smaller. This difference also demonstrates the supression of collagen type I expression in cultures on aggrecan. Thus, it is important to look for chondrocyte-specific markers when evaluating the extent of differentiation of the fibroblasts.

The dermal fibroblast cells in culture on aggrecan do initiate expression of chondrocyte markers such as aggrecan and collagen type II as seen at both the protein and mRNA level. Interestingly, there appears to be an “age” effect with these cells in culture. With greater passage number, the cellular response is decreasingly robust. Although IGF-1 is highly effective at priming the cells for the response to aggrecan, with the older cells, such as those present after 40+ passages, it was observed that increased numbers of cells form monolayers at the later time points compared to the lower passage numbers. Accordingly, it is preferred to harvest the redifferentiated cells for use before the cultures have been passaged to such an extent that the cells are no longer robust. It was also observed that cells freshly isolated from rabbit skin require no IGF-1 to initiate either the aggregate-forming response to aggrecan or the expression of proteoglycans (data not shown).

Although it was observed that the Rab9 cells diminish their response with increased passage number, the formation of aggregates does still occur and these aggregates are expressing collagen type II and aggrecan, as seen by antibody detection, staining and RT-PCR. The morphology of the cells is also different as seen in the compact aggregates formed. While a monolayer can form after several days in culture in aggrecan, the initial reaction of the cells is to adopt a rounded morphology, optimal for forming large nodules of cells. The intense staining of aggregates with Safranin O is in sharp contrast with the absence of staining in the monolayer cultures on plastic. While the GAG concentrations as determined by the DMMB assay do not duplicate such a dramatic result, it may be that the large number of monolayer cells in the culture diluted the effects of high proteoglycan synthesis.

Likewise, with the total collagen assay, cells in monolayers will secrete collagen type I and/or IV as their matrix proteins of choice. If small nodules in the culture are secreting elevated amounts of collagen type II, it will be difficult to determine in such a large pool of cells. Better measures of collagen type II expression are the antibody staining and the RT-PCR analysis. As seen in the RT-PCR results, these cultures are still expressing collagen type I. While one could argue that these cells are not becoming chondrocytes, rather they are fibrochondrocytes, this is not yet clear. Since the RNA is isolated on a total-well basis, the monolayer cells are also included in these cultures and will contribute to the RNA profile of the culture. In instances where there is a large monolayer population, it may present a more accurate picture of the cells to remove the aggregates from the plate prior to lysis for RNA isolation and collect aggregate RNA separately.

### Example 2

Redifferentiation of Human Foreskin Fibroblasts to Cartilage-Like Cells

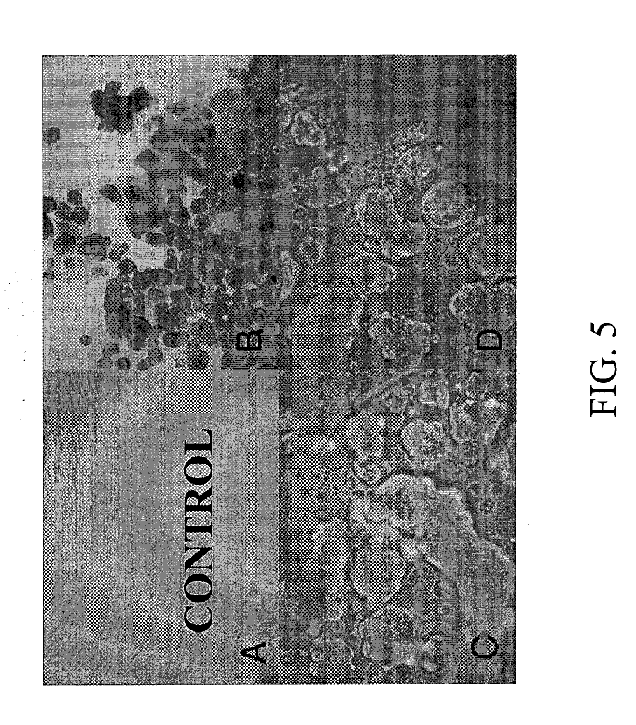

The human foreskin fibroblast line Hs27 was cultured as described in Example 1 and the redifferentiated cells were evaluated in the same way. The results for these cells were similar to those observed with the rabbit dermal fibroblasts. FIGS. 5A-D are photomicrographs showing the morphology of human foreskin cells after 24 hours in culture with or without aggrecan. FIG. 5A is a control culture in which the cells were grown on the uncoated plastic tissue culture plate (at 40× magnification). FIG. 5B shows the morphology of a corresponding culture grown on the aggrecan-coated plate. FIGS. 5C and 5D show the aggrecan-promoted cells of FIG. 5B at 40× and 100×, respectively. The cell aggregates in the aggrecan-treated cultures resemble in vitro cartilage development in chondrocytes.

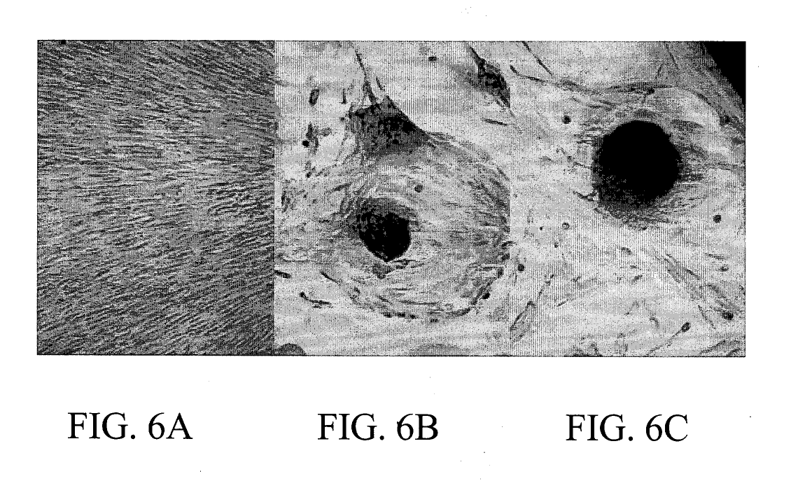

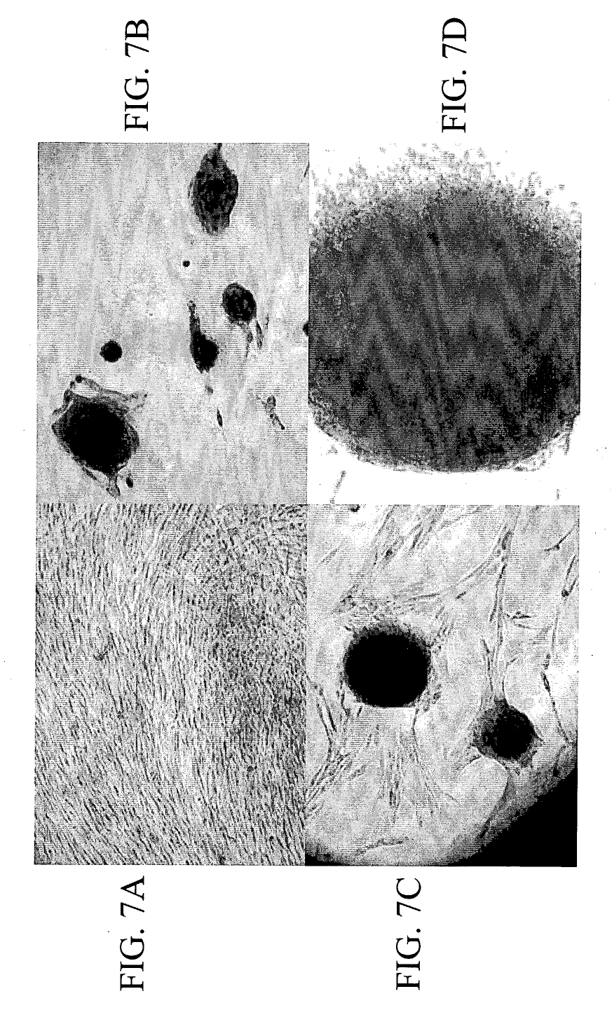

After one week in culture, the aggrecan-treated and the control (untreated) foreskin fibroblasts were stained with Safranin O to detect proteoglycan markers characteristic of cartilage tissue, as described above. FIG. 6A is a photomicrograph showing negligible staining of the control cells, while FIG. 6B shows marked clustering of the cells grown on aggrecan and dark staining of the proteoglycan. FIG. 6C is a photomicrograph of an aggregate similar to the one shown in FIG. 6B and taken at higher magnification (200×). FIGS. 7A-D show the results of an assay for proteoglycan in another series of aggrecan-containing and control cultures after one week in culture. FIG. 7A shows the lightly stained control fibroblasts. FIGS. 7B-C are photomicrographs showing concentrated staining of cell aggregates at (100×), (100×) and (200×), respectively.

The results of the antibody staining assays for collagen type II production by cells after one week of in culture are shown in FIGS. 8A-D. FIG. 8A is a photomicrograph of a control plate. FIG. 8B shows the aggrecan-grown cells without antibody staining (100×). FIGS. 8C and 8D show cell culture plates like those in FIG. 8B except the collagen type II is revealed by antibody staining.

Tissue Engineering Applications

The above-described cells, compositions and methodologies offer several new clinical options for cartilage repair and replacement and constitute significant technological advancements in the provision of tissue engineered cartilage. For example, for a patient diagnosed with a focal lesion on the surface of his articular cartilage, the doctor could obtain a piece of skin, from which fibroblasts would be obtained, culture those fibroblasts as described above such that the cell numbers expanded sufficiently, and then seed them on a suitable scaffold coated with cartilage matrix proteoglycans. The autologous redifferentiated cells would subsequently be implanted in the patient at the site of the cartilage defect that is in need of repair. This scenario would be minimally invasive for the patient, would provide a rapidly dividing cell source for tissue regeneration, and would provide also provide the environmental factors needed to drive the chondrogenic differentiation of the cells. Such “custom made” autologous cartilage-like materials would also most likely avoid any immune reactions that might potentially occur with non-autologous implant materials.

Another way in which the above-described redifferentiated fibroblast cells and procedures are expected to meet a widespread medical need is in the provision and use of “off the shelf” compositions containing allogenic fibroblast cells. For example, donor fibroblast cells can be processed and cultured in advance of need, formed into predetermined two-dimensional or three-dimensional configurations. Then an appropriately sized piece (e.g., a sheet or a plug) can be provided to the medical practitioner in accordance with a particular patient's need. At the time of surgery, the practitioner can prepare the site of the articular cartilage defect to receive a correspondingly sized piece containing the living redifferentiated fibroblasts. In this way the surgeon can prepare the site and implant the cartilage replacement piece at the same time.

While preferred embodiments of this invention have been shown and described, modifications thereof can be made by one skilled in the art without departing from the spirit or teaching of this invention. The embodiments described herein are exemplary only and are not limiting. Many variations and modifications of the system and apparatus are possible and are within the scope of the invention. For example, although dermal fibroblasts are highly preferred as the source of the redifferentiated cells because of their abundance and easy availability, it is expected that cartilage tissue could be successfully substituted for fibroblasts as the source of cells. It is expected that cartilage cells will be prevented from losing their characteristic chondrocyte properties when cultured as described herein. Likewise, it is expected that other fully differentiated cells, including smooth muscle cells, fat cells, tendon cells, ligament cells, and others, are capable of being redifferentiated to chondrocyte-like cells using similar procedures. Accordingly, the scope of protection is not limited to the embodiments described herein, but is only limited by the claims which follow, the scope of which shall include all equivalents of the subject matter of the claims.

What is claimed is:

1. A redifferentiated fibroblast cell that exhibits at least one characteristic of a chondrocyte, wherein a proteoglycan other than perlecan was used to induce differentiation of said cell.

2. The cell of claim 1 wherein the fibroblast cell is a dermal fibroblast cell.

3. The cell of claim 1 wherein said at least one characteristic comprises expression of collagen type II and/or mRNA encoding of said collagen type II.

4. The cell of claim 1 wherein said at least one characteristic comprises expression of at least one cartilage proteoglycan marker.

5. The cell of claim 3 wherein said at least one proteoglycan marker comprises a glycosaminoglycan.

6. The cell of claim 1 wherein said at least one proteoglycan comprises aggrecan.

7. The cell of claim 1 wherein said cell differentiated from said fibroblast along the chondrogenic lineage.

8. A composition for treatment of a cartilage defect or disorder comprising the cell of claim 1.

9. The composition of claim 8 comprising a three-dimensional structure.

10. A kit for treatment of a cartilage defect comprising the composition of claim 8 wherein said cell is allogenic to an individual in need of such treatment.

11. A method of treating a cartilage defect or disorder comprising implanting the composition of claim 8 into an individual in need there of at a site comprising a cartilage defect.

12. The method of claim 11 wherein said cell is autologous to said individual.

13. The method of claim 11 wherein said cell is allogenic to said individual.

14. The method of claim 11 comprising preparing said defect site to receive said three-dimensional structure.

15. The method of claim 11 comprising resurfacing an articular cartilage defect with said composition.

16. A method of making the cell of claim 1 comprising:
culturing a fibroblast cell on a surface coated with a proteoglycan other than perlecan, and,
optionally, coating a three-dimensional scaffold with said proteoglycan and seeding the resulting proteoglycan-coated scaffold with said fibroblast cell.

17. The method of claim 16 wherein said proteoglycan is aggrecan.

18. The method of claim 16 wherein said culturing comprises treating said fibroblast cell with at least one chondrogenic growth factor or cytokine.

19. The method of claim 16 wherein said culturing comprises treating said fibroblast cell with IGF-1.

20. The method of claim 16 wherein said culturing comprises exposing said fibroblast cell to a medium comprising at least one growth factor.

21. The method of claim 16 comprising detecting a chondrocytic phenotype in said cultured cell.

22. The method of claim 16 wherein said fibroblast cell is a dermal fibroblast cell.

23. A method of inducing chondrogenesis in a differentiated cell comprising culturing said differentiated cell on a surface containing at least one cartilage-derived proteoglycan other than perlecan.

24. The method of claim 23 wherein the differentiated cell is selected from the group consisting of fibroblasts, muscle cells, fat cells, tendon cells, ligament cells and chondrocytes.
